(19) United States
(12) Plant Patent Application Publication
(10) Pub. No.: US 2004/0181839 P1
(43) Pub. Date: Sep. 16, 2004

(54) Dieffenbachia hybrid called 'Tropic Honey'

(76) Inventor: Richard J. Henny, Apopka, FL (US)

Correspondence Address: Thomas D. Stadsklev, Florida Foundation Seed Producers, Inc., P.O. Box 309, Greenwood, FL, 32443, US

(21) Appl. No.: 10/386,175

(22) Filed: Mar. 11, 2003

(51) Int. Cl. A01H 5/00

(52) U.S. Cl. PLT/378

(57) ABSTRACT

A new and distinct cultivar of Dieffenbachia that is characterized by its large size and excellent branching and undulating golden yellow leaves with dark green borders that are held close to the main stem to produce a compact plant.

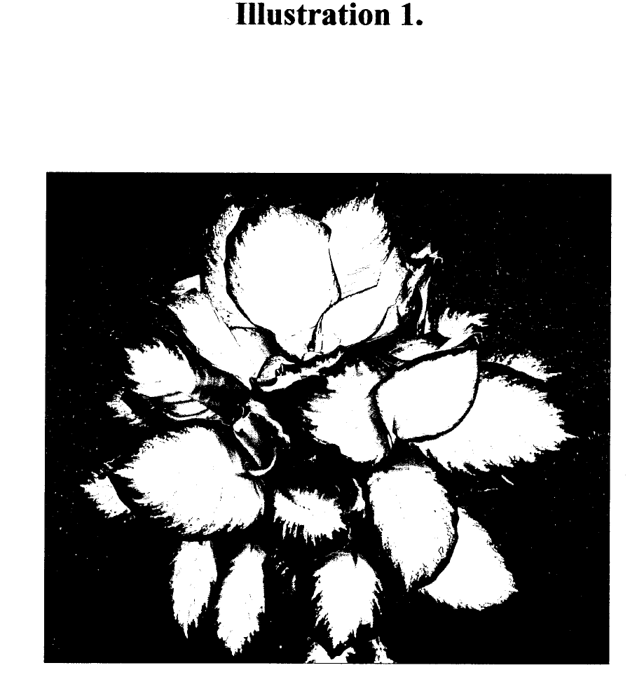

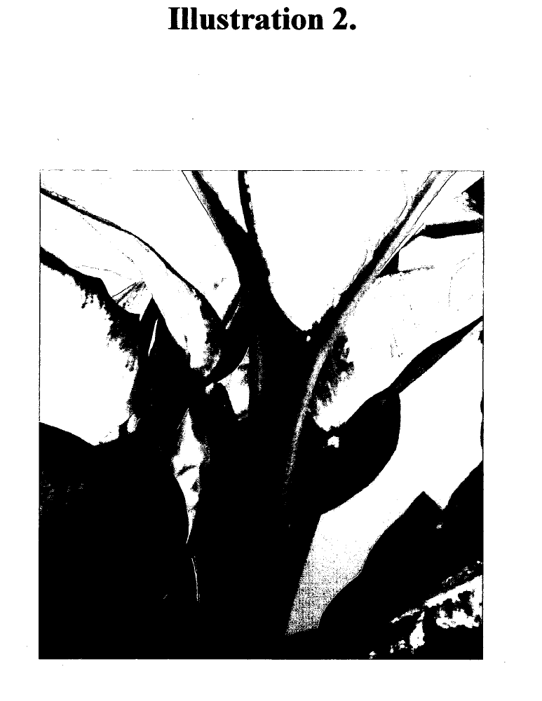

A new and distinct dieffenbachia hybrid whose novelty consist of the following unique combination of features.

1. The leaves are deep golden yellow and are highlighted by a dark green border.

2. The leaves are very thick with sunken veins that produce a wavy but uniform texture.

3. There is a distinct undulation in the leaf margins that results in added depth to the leaf coloration.

4. The petiole wings on the main shoot extend all the way to the base of the leaf blade to give a very compact appearance as the leaf blade hugs the main stem.

5. Plants are large, compact and well-branched averaging 4-8 basal shoots per plant.

The present invention comprises a new and distinct cultivar of Dieffenbachia, botanically known as Dieffenbachia hybrida, and referred to by the cultivar name Tropic Honey.

The new cultivar is a product of a planned breeding program carried out by the inventor Richard Henny in Apopka, Fla. Dr. Henny has performed many crosses with Dieffenbachia since 1980 as part of his extensive ornamental tropical foliage plant breeding program.

The new cultivar is a product of cross-pollination between Dieffenbachia ‘Victory’ (pollen parent) and Dieffenbachia ‘Tropic Marianne’ (U.S. Plant Pat. No. 8,832; seed parent).

The cultivar was discovered from the progeny of the stated cross by Richard J. Henny in Apopka, Fla. It was selected because of its vigorous growth, fairly large size, compact habit and undulating leaves that displayed a bright golden color accented by a narrow green border. Asexual propagation by cuttings, first performed by Richard J. Henny in Apopka, Fla., was used to increase the number of plants for evaluation. Subsequently, plants were propagated by tissue culture that also has demonstrated the stability of the combination of characteristics of Tropic Honey from generation to generation.

The following observations, measurements and values describe plants grown in Apopka, Fla., under greenhouse conditions which closely approximate those generally used in horticultural practice.

The following traits have been repeatedly observed to be characteristics that in combination distinguish Tropic Honey from other Dieffenbachia of the same general type such as the well-known cultivars Tropic Marianne and Camille.

Tropic Honey differs from Tropic Marianne in the following ways.

1. The leaves of Tropic Honey are very thick with sunken veins that produce a wavy but uniform texture. Tropic Marianne leaves are smooth.

2. There is a distinct undulation in Tropic Honey leaf margins that results in added depth to the leaf coloration. Tropic Marianne leaves lack such undulation.

3. The petiole wings on the main shoot of Tropic Honey extend all the way to the base of the leaf blade to give a very compact appearance as the leaf blade hugs the main stem. Tropic Marianne petiole wings stop within 4-7 cm of the base of the leaf blade.

4. Leaves on the main stem of Tropic Honey are held closer to the stem to give a more compact appearance compared to Tropic Marianne.

Tropic Honey differs from Camille in the following ways.

1. Tropic Honey is much larger and more vigorous than Camille.

2. Tropic Honey leaves are thicker and much bigger than Camille.

3. Tropic Honey does not branch as much as Camille.

All color references are measured against The Royal Horticultural Society Color Chart. Colors are approximate as color depends on horticultural practices such as light level and fertilization rate, among others, without, however any variance in genotype.

### BRIEF DESCRIPTION OF THE DRAWINGS

The two color photographic drawings were taken from a typical plant of Dieffenbachia Tropic Honey grown in a 20 cm diameter pot (3.9 liter volume), approximately 24 weeks after planting a 12-week-old liner obtained by tissue culture and grown under appropriate growing conditions. Colors are as accurate as possible with color illustrations of this type.

1. The first drawing depicts a top perspective view of a plant of ‘Tropic Honey’ showing the golden foliar variegation and the compact foliage canopy due to the short thick clasping petioles. The regular undulations in the leaf margins are clearly visible.

2. The second drawing depicts the upper and lower leaf surface and the main stem and petiole coloration.

Origin: Seedling selected from a cross of Dieffenbachia ‘Victory’ (pollen parent) and Dieffenbachia ‘Tropic Marianne’ (U.S. Plant Pat. No. 8,832; seed parent).

Classification:

Propagation: Asexual propagation either by tissue culture, stem cuttings or division.

Plant: In a 20 cm diameter pot for a plant grown from a 12 week old liner after 20 weeks under appropriate growing conditions, Tropic Honey has a canopy height, from the soil surface of 30 to 33 cm and a canopy width of 47 to 51 cm. Plants of this age average 4 to 5 basal shoots. The petioles remain closely aligned to the main stem and result in a very compact appearance for the foliage canopy compared to other dieffenbachia cultivars of similar size.

Stem:

Growth pattern.—The stem is erect in growth and is 2.5 to 3.0 cm in diameter five (5) cm above the soil surface. Internode distance is approximately 1.7 cm to 2.0 cm at five (5) cm above the soil.

Color.—The stem color is green 137B/C. The color is uniform with no mottling.

Petiole: The following information is based on the 3rd expanded leaf from the apex.

Dimensions.—The petioles are approximately 14.0 to 17.0 cm in length. The petiole has fleshy edges extending from the midrib and referred to as wings. The wings are approximately 8 mm to 12 mm wide one-half the distance from the petiole base to the wing apex. The wings extend from the base of the petiole all the way to the leaf base. On lateral basal shoots the petiole wings do not reach all the way to the leaf base and may stop 2 to 6 cm short of the leaf base. The petiole follows the stem axis to within 1.0 to 2.0 cm of the leaf base. The petiole and wings average approximately 7 mm to 10 mm in diameter one-half the distance between the top of the wing and the base of the leaf.

Color.—The petiole and petiole wings are green 137 A/B on the outer edges extending inward about ⅓ the petiole width from each edge. The central portion of the petiole is lighter yellow-green 146B/C. The inner petiole consists of three colors. The outer margins are green 137B and at approximately half way to the center blend to a lighter green-white 157A that extends down the petiole center. At the junction of the petiole and leaf blade there is a triangular-shaped area approximately 0.5 cm on each edge that also is green-white 157A. This area is bordered by the outer petiole that is darker green 137A/B.

Leaf:

Growth pattern.—The leaf is oblong with a cuspidate apex and cordate base. The margin is entire. The leaf is asymmetric with the side of the leaf unrolling first having less surface area than the side unrolling last. The leaf is oriented parallel to the stem axis at the time of full unrolling, changing to approximately 40-50 degrees above perpendicular to the stem axis as more leaves unroll above it. The midrib is straight over two-thirds the length of the leaf and curved downward toward the tip. The leaf blade undulates along the margin. The leaf is thick and somewhat puckered. The leaf blades are angled upward from the midrib but flatten as the leaf ages.

Dimensions.—For the pot size and growing time indicated, the largest leaf is approximately 36-38 cm long and approximately 15-16 cm wide. An average sized leaf is approximately 35-36 cm long and approximately 14-15 cm wide.

Midrib.—The leaf midrib is thick and prominent and ranges from a width of 1 cm at the base of the leaf to approximately 0.7 to 0.8 cm at a distance equally between the leaf base and the leaf tip. At the base of the leaf, the midrib is green 137A/B. Within 1.0 to 2.0 cm the leaf base, the green coloration begins to lighten due to convergence with several small parallel green-white 157A/B stripes, and at approximately half way to the leaf tip the green becomes very faint and is hardly visible at the leaf tip.

Primary veins.—The primary veins are sunken into the upper leaf surface and protrude from the lower surface. The primary veins are the same color 157A/B as the leaf midrib and do not contain any green coloration.

Color and pattern.—Leaf color pattern is uniform beginning with a green 137A/B border that is 0.5 to 1.5 cm in width edges mature leafblades on the upper and lower surfaces. The green border lightens to yellow-green 154D in the central portion of the leaf blade. This color lightens to yellow-green 150D surrounding the midrib and primary veins. The leaf midrib lightens even more to a green-white 157A/B.

Axillary breaks: In our experimental growth tests plants produced approximately 4-8 axillary breaks with at least one leaf expanded.

Inflorescence: Typical of Dieffenbachia and does not have commercial significance.

Roots: Moderately thick white roots with fine laterals.

I claim:

1. A new and distinct Dieffenbachia hybrid as illustrated and described. It is characterized by leaves that are deep a golden yellow and are highlighted by a dark green border, leaves that are very thick with sunken veins that produce a wavy but uniform texture with a distinct undulation in the margins that results in added depth to the leaf coloration. The petiole wings on the main shoot extend all the way to the base of the leaf blade to give a very compact appearance as the leaf blade hugs the main stem. Plants are large, compact and well-branched averaging 4-8 basal shoots per plant.
